(19) United States
(12) Patent Application Publication
(10) Pub. No.: US 2026/0047861 A1
(43) Pub. Date: Feb. 19, 2026

(54) BALLOON FOR BALLOON CATHETER AND BALLOON CATHETER INCLUDING SAME

(71) Applicant: KANEKA CORPORATION, Osaka-shi, Osaka (JP)

(72) Inventors: Masahiro KOJIMA, Settsu-shi (JP); Takahisa HAMABUCHI, Osaka-shi (JP); Yoshinori NAKANO, Settsu-shi (JP); Masato TSUEDA, Settsu-shi (JP)

(73) Assignee: KANEKA CORPORATION, Osaka-shi, Osaka (JP)

(21) Appl. No.: 19/104,010

(22) PCT Filed: Jul. 26, 2023
(86) PCT No.: PCT/JP2023/027370

§ 371 Date: Feb. 14, 2025

(30) Foreign Application Priority Data
Aug. 24, 2022 (JP) 2022-133113

(51) Int. Cl. A61B 17/3207; A61B 17/00; B29C 33/42; B29C 49/48; B29L 31/00

(52) U.S. Cl. CPC A61B 17/320725; B29C 33/42; B29C 49/48; A61B 2017/00557; B29L 2031/7543

(57) ABSTRACT

Disclosed is a balloon for a balloon catheter that can enhance non-slip performance to prevent the balloon from slipping off the lesion and improve scoring performance for biting into stenotic portions. A balloon (2) for a balloon catheter having a balloon body (20) and a protrusion part (28), the balloon body (20) and the protrusion part (28) are composed of the same material, and a ratio Ra₁/Ra₂ of a surface roughness Ra₁ of the protrusion part (28), measured along a reference length in a direction (a1) parallel to the longitudinal axis direction (x1), to a surface roughness Ra₂ of the protrusion part (28), measured along a reference length in a direction (a2) perpendicular to the longitudinal axis direction (x1), being greater than 1.

### TECHNICAL FIELD

The present invention relates to a balloon for a balloon catheter and a balloon catheter including the same.

### BACKGROUND ART

Diseases such as angina pectoris and myocardial infarction are caused by the formation of a stenotic portion hardened by calcification and other factors in inner walls of blood vessels. One of the treatments for these diseases is angioplasty, in which a balloon catheter is used to dilate the stenotic portion. The angioplasty is a minimally invasive therapy that does not require an open chest procedure like bypass surgery and is widely used.

In the angioplasty, conventional balloon catheters may have difficulties in dilating stenotic portions hardened by calcification and other factors. Alternatively, a method is also used to dilate the stenotic portions by placing a stent, a device that expands the stenotic portions, but in some cases, for example, after this treatment, a neointima of the blood vessel proliferates excessively and the blood vessel narrows again, causing an ISR (In-Stent-Restenosis) lesions may occur. The neointima in ISR lesions is soft and the surface is slippery, so a standard balloon catheter may cause the balloon to shift out of the lesion site during balloon dilation to damage the vessel.

As balloon catheters that can dilate a stenosis even in such calcified or ISR lesions, balloon catheters with a protrusion, blade or scoring element on the balloon to bite into the stenosis have been developed. For example, Patent document 1 discloses a balloon having a protrusion, and a method for producing a balloon which includes a step of forming a protrusion by welding at least a portion of the adjacent inner surfaces to each other at the part where the inner surfaces of the balloon are arranged facing each other. Patent document 2 discloses a balloon having a gather, which is a protrusion part, and a method of forming a gather on a balloon using a mold.

### RELATED ART DOCUMENT

### Patent Document

Patent document 1: JP 2017-12678 A

Patent document 2: JP 2005-511187 T

### SUMMARY OF THE INVENTION

### Problems to be Solved by the Invention

However, in the conventional balloons described above, while the protrusion part may come into contact with the stenotic portion, it may insufficiently penetrate into hardened calcified lesions. In addition, the balloon may be difficult to secure to lesions such as ISR lesions, which have slippery surfaces, causing the balloon to slip out of position. As a result, the intended site may not be incised, or unintended damage may be caused to blood vessels outside the treatment area.

In view of the above circumstances, the objective of the present invention is to provide a balloon for a balloon catheter that can enhance non-slip performance to prevent the balloon from slipping off the lesion and improve scoring performance for biting into stenotic portions, as well as a balloon catheter equipped with the balloon.

### Means for Solving the Problems

A balloon for a balloon catheter in accordance with an embodiment of the present invention that can solve the above problem is as follows.
[1] A balloon for a balloon catheter having a longitudinal axis direction and a radial direction, comprising: a balloon body having an outer surface and inner surface; and a protrusion part that projects outward from the outer surface of the balloon body in the radial direction and extends in the longitudinal axis direction, wherein the balloon body and the protrusion part are composed of the same material; and a ratio Ra₁/Ra₂ of a surface roughness Ra₁ of the protrusion part, measured along a reference length in a direction parallel to the longitudinal axis direction, to a surface roughness Ra₂ of the protrusion part, measured along a reference length in a direction perpendicular to the longitudinal axis direction, is greater than 1.

The balloon for a balloon catheter in accordance with embodiments of the present invention is preferably any one of the following [2] to [7].
[2] The balloon for a balloon catheter according to [1], wherein in a cross-section perpendicular to the longitudinal axis direction, the protrusion part has a tip region including an outer end in the radial direction and a base region located inward from the tip region in the radial direction, a surface roughness of the base region is smaller than a surface roughness of the tip region where a surface roughness of the protrusion part is measured along a reference length in a direction parallel to the longitudinal axis direction; and a surface roughness of the base region is smaller than a surface roughness of the tip region where a surface roughness of the protrusion part is measured along a reference length in a direction perpendicular to the longitudinal axis direction.
[3] The balloon for a balloon catheter according to [1] or [2], wherein in a cross-section perpendicular to the longitudinal axis direction, the protrusion part has a tip region including an outer end in the radial direction and a base region located inward from the tip region in the radial direction; a ratio Ra₁₍ₜᵢₚ₎/Ra₂₍ₜᵢₚ₎ of a surface roughness Ra₁₍ₜᵢₚ₎, where a surface roughness of the tip region is measured along a reference length in a direction parallel to the longitudinal axis direction, to a surface roughness Ra₂₍ₜᵢₚ₎, where a surface roughness of the tip region is measured along a reference length in a direction perpendicular to the longitudinal axis direction, is greater than 1; and a ratio Ra_{1(base)}/Ra_{2(base)} of a surface roughness Ra_{1(base)}, where a surface roughness of the base region is measured along a reference length in a direction parallel to the longitudinal axis direction, to a surface roughness Ra_{2(base)}, where a surface roughness of the base region is measured along a reference length in a direction perpendicular to the longitudinal axis direction, is greater than 1.
[4] The balloon for a balloon catheter according to [2] or [3], wherein a surface roughness of the balloon body is greater than the surface roughness of the base region where the surface roughness of the base region and the surface roughness of the balloon body are measured along a reference length in a direction perpendicular to the longitudinal axis direction.
[5] The balloon for a balloon catheter according to any one of [2] to [4], wherein a surface roughness of the balloon body is greater than the surface roughness of the tip region where the surface roughness of the tip region and the surface roughness of the balloon body are measured along a reference length in a direction perpendicular to the longitudinal axis direction.
[6] The balloon for a balloon catheter according to any one of [2] to [5], wherein a surface roughness of the balloon body is smaller than the surface roughness of the tip region where the surface roughness of the tip region and the surface roughness of the balloon body are measured along a reference length in a direction parallel to the longitudinal axis direction.
[7] The balloon for a balloon catheter according to any one of [2] to [6], wherein a surface roughness of the balloon body is smaller than the surface roughness of the base region where the surface roughness of the base region and the surface roughness of the balloon body are measured along a reference length in a direction parallel to the longitudinal axis direction.

The present invention also provides the following.
[8] A balloon catheter comprising the balloon for a balloon catheter according to any one of the above [1] to [7].

### Effects of the Invention

According to the above-described balloon for a balloon catheter and the balloon catheter, the protrusion part provided on the outer surface of the balloon can improve non-slip performance, making it less likely for the balloon to slip from the lesion, while also enhancing scoring performance by allowing the protrusion part to penetrate stenotic portions. This enables efficient dilation of the stenotic portions and facilitates safe treatment by reducing the risk of damaging blood vessels outside the target treatment area.

### BRIEF DESCRIPTION OF THE DRAWINGS

FIG. 1 is a side view of a balloon catheter in accordance with one embodiment of the present invention.

FIG. 2 is a perspective view of a balloon for a balloon catheter in accordance with one embodiment of the present invention.

FIG. 3 is a III-III cross-sectional view of FIG. 1.

FIG. 4 is a cross-sectional view showing a variation of FIG. 3.

FIG. 5 is a roughness curve obtained by measuring the surface roughness of a protrusion part of a balloon for a balloon catheter in accordance with one embodiment of the present invention along a reference length in a direction parallel to the longitudinal axis direction.

FIG. 6 is a roughness curve obtained by measuring the surface roughness of the protrusion part of the balloon for a balloon catheter used in the measurement of FIG. 5 along a reference length in a direction perpendicular to the longitudinal axis direction.

FIG. 7 is a perspective view of a parison in accordance with one embodiment of the present invention before inflation.

FIG. 8 is a cross-sectional view in the longitudinal axis direction of a mold in accordance with one embodiment of the present invention.

FIG. 9 is a IX-IX cross-sectional view of FIG. 8.

### MODE FOR CARRYING OUT THE INVENTION

Hereinafter, the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of the present invention, the dimensions may differ from the actual dimensions in some cases.

### 1. Balloon for Balloon Catheter

A balloon for a balloon catheter in accordance with embodiments of the present invention is a balloon for a balloon catheter having a longitudinal axis direction and a radial direction, having a balloon body having an outer surface and an inner surface; and a protrusion part that projects outward from the outer surface of the balloon body in the radial direction and extends in the longitudinal direction, wherein the balloon body and the protrusion part are composed of the same material; and a ratio Rat/Raz of a surface roughness Rai, measured along a reference length in a direction parallel to the longitudinal axis direction, to a surface roughness Raz, measured along a reference length in a direction perpendicular to the longitudinal axis direction, is greater than 1.

Dilation of the stenosis with a balloon catheter is performed by delivering the balloon, which is provided at the distal end part of the balloon catheter, to the stenosis and then inflating the balloon to incise the stenosis by allowing the protrusion part disposed on the outer surface of the balloon body to bite into the stenosis. At this time, the greater the surface roughness of the protrusion part in a direction parallel to the longitudinal axis direction of the balloon, i.e., the direction parallel to the balloon's travel direction within the blood vessel, the greater the resistance of the protrusion part to the balloon's travel direction, and thus, the protrusion part can improve the non-slip performance, which prevents it from deviating from the intended position. On the other hand, the smaller the surface roughness of the protrusion part in a direction perpendicular to the longitudinal axis direction of the balloon, i.e., the direction in which the protrusion part enters the stenosis, the smaller the resistance of the protrusion part against penetration into the stenosis, and the easier it is for the protrusion part to bite into the stenosis, so the scoring performance can be improved. Accordingly, it is possible to make a balloon with non-slip and scoring performance by making the ratio Ra₁/Ra₂ of a surface roughness Ra₁ of the protrusion part, measured along a reference length in a direction parallel to the longitudinal axis direction, to a surface roughness Ra₂ of the protrusion part, measured along a reference length in a direction perpendicular to the longitudinal axis direction, greater than 1.

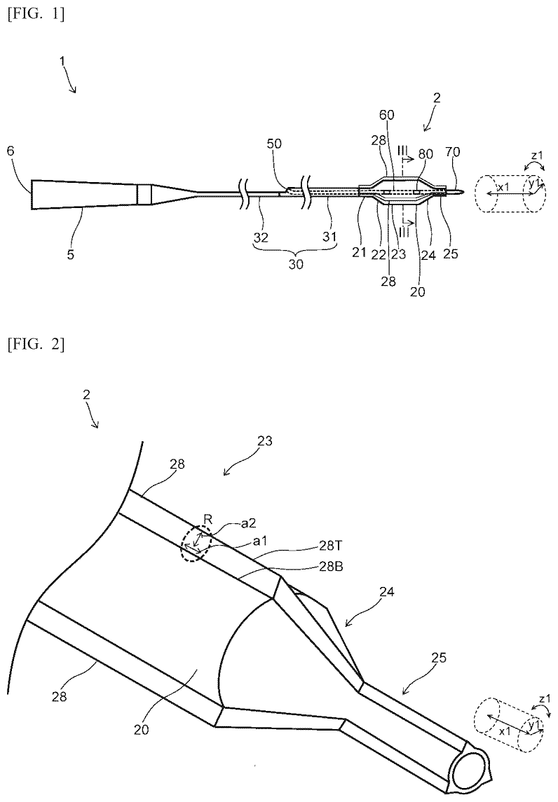

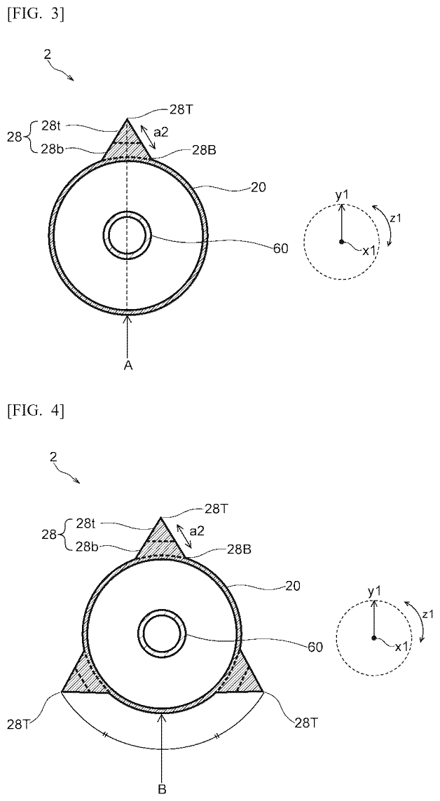

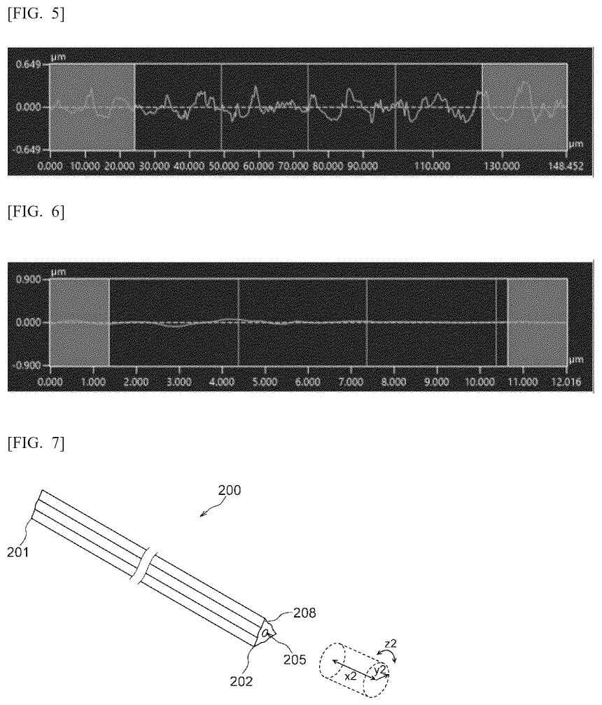

In this specification, the balloon for a balloon catheter may simply be referred to as “the balloon.”Hereinafter, a balloon for a balloon catheter in accordance with embodiments of the present invention will be described, referring to FIG. 1 to FIG. 6. FIG. 1 is a side view of a balloon catheter in accordance with one embodiment of the present invention. FIG. 2 is a perspective view of a balloon for a balloon catheter in accordance with one embodiment of the present invention, showing the distal side of the balloon. FIG. 3 is a III-III cross-sectional view of FIG. 1, showing a cross-section perpendicular to the longitudinal axis direction of a balloon for a balloon catheter in accordance with an embodiment of the present invention. FIG. 4 is a cross-sectional view showing a variation of FIG. 3. FIG. 5 is a roughness curve obtained by measuring the surface roughness of a protrusion part of a balloon for a balloon catheter in accordance with one embodiment of the present invention along a reference length in a direction parallel to the longitudinal axis direction, and FIG. 6 is a roughness curve obtained by measuring the surface roughness of the protrusion part of the balloon for a balloon catheter used in the measurement of FIG. 5 along a reference length in a direction perpendicular to the longitudinal axis direction.

As shown in FIG. 1, a balloon 2 is used for a balloon catheter 1. The balloon 2 is connected to a distal end part of a distal shaft 31, and the balloon 2 can be inflated by introducing a fluid through a lumen of the distal shaft 31 and the balloon 2 can be deflated by discharging the fluid. To control the inflation and deflation of the balloon 2, the fluid can be introduced or discharged using an indeflator (pressurizer for a balloon). The fluid may be a pressurized fluid that is pressurized by a pump or the like. The balloon catheter 1 will be described in detail in the section “2. balloon catheter.”

The balloon 2 has a longitudinal axis direction x1, a circumferential direction z1 along the outer edge of the balloon 2 in a cross-section perpendicular to the longitudinal axis direction x1, and a radial direction y1 connecting the figure center of the outer edge of the balloon 2 and a point on the outer edge in a cross-section perpendicular to the longitudinal axis direction x1. In this specification, the direction of the user's hand side in the longitudinal axis direction x1 is referred to as a proximal side, and the direction opposite the proximal side, i.e., the direction of the treatment target side, is referred to as a distal side.

The components and parts other than the balloon 2 have their own longitudinal axis direction, radial direction, and circumferential direction, which may be the same as or different from the longitudinal axis direction x1, radial direction y1, and circumferential direction z1 of the balloon 2. In this specification, for the sake of ease of understanding, all components and parts are described as having the same longitudinal axis direction, radial direction, and circumferential direction as the longitudinal axis direction x1, radial direction y1, and circumferential direction z1 of the balloon 2.

The balloon 2 has a proximal end and distal end in the longitudinal axis direction x1, and as shown in FIG. 1, preferably has a straight tubular part 23, a proximal tapered part 22 located proximal to the straight tubular part 23, a proximal sleeve part 21 located proximal to the proximal tapered part 22, a distal tapered part 24 located distal to the straight tubular part 23, and a distal sleeve part 25 located distal to the distal tapered part 24. The straight tubular part 23 preferably has a roughly cylindrical shape having approximately the same diameter in the longitudinal axis direction x1, but it may have a different diameter in the longitudinal axis direction x1. The proximal tapered part 22 and the distal tapered part 24 are preferably formed into a conical shape by reducing the diameter as they are apart from the straight tubular part 23. Since the straight tubular part 23 has the largest diameter, when the balloon 2 is inflated at a lesion site, such as a stenosis, the straight tubular part 23 can make sufficient contact with the lesion, make it easier to perform treatments such as dilation of the lesion. In addition, since the proximal tapered part 22 and the distal tapered part 24 are reduced in diameter, when the balloon 2 is deflated, the outer diameter of the proximal and distal end parts of the balloon 2 can be decreased, reducing the step between the distal shaft 31 and the balloon 2, thereby facilitating the insertion of the balloon 2 into the body cavity.

Preferably, while the proximal tapered part 22, the straight tubular part 23, and the distal tapered part 24 are the parts that inflate when a fluid is introduced into the balloon 2, the proximal sleeve part 21 and the distal sleeve part 25 are not inflated. At least part of the proximal sleeve part 21, which does not inflate, can be fixed to the distal shaft 31, and at least part of distal sleeve part 25 can be fixed to an inner shaft 60.

The balloon 2 has a balloon body 20 having an outer surface and an inner surface, and a protrusion part 28 that projects outward from the outer surface of the balloon body 20 in the radial direction y 1 and extends in the longitudinal axis direction x1.

The balloon body 20 defines the basic shape of the balloon 2, and the protrusion part 28 is provided on the outer surface of the balloon body 20 in any pattern, such as linear, dot, mesh, or spiral patterns. By providing the protrusion part 28 on the outer surface of the balloon body 20, the protrusion part 28 can be provided with a scoring function, and it becomes possible to crack and dilate calcified stenotic portions in angioplasty. In addition, the protrusion part 28 can also contribute to improving the strength of the balloon 2 and preventing over-inflation during being pressurized.

The protrusion part 28 is preferably provided in the straight tubular part 23. The protrusion part 28 provided in the straight tubular part 23, which is most easily contacts the lesion, enables easy dilation of the stenosis.

As shown in FIG. 1 and FIG. 2, the protrusion part 28 may be provided in the entire area of the balloon 2 in the longitudinal axis direction x1, i.e., in the straight tubular part 23, tapered parts, and sleeve parts. By providing the protrusion part 28 in portions other than the straight tubular part 23, the strength of the balloon 2 can be enhanced, and the effect of suppressing over-inflation during being pressurized can be improved. Alternatively, although not shown in the figures, the protrusion part 28 may be provided in the straight tubular part 23, while either not being provided in the tapered and sleeve parts or being provided at a lower height than in the straight tubular part 23. It also may be provided in at least a part of the tapered and sleeve parts. By having areas in the tapered and sleeve parts where the protrusion part 28 is either absent or provided at a lower height than in the straight tubular part 23, the insertion performance of the balloon 2 can be improved.

The protrusion part 28 is preferably made of the same material as the balloon body 20, and more preferably, the balloon body 20 and the protrusion part 28 are integrally molded. By forming the balloon body 20 and the protrusion part 28 from the same material, it becomes possible to prevent the protrusion part 28 from damaging the outer surface of the balloon body 20 while maintaining the flexibility of the balloon 2. By integrally forming the balloon body 20 and the protrusion part 28, detachment of the protrusion part 28 from the balloon body 20 can be prevented. Alternatively, when there is a certain degree of compatibility with the material used to form the balloon body 20, the material for forming the protrusion part 28 may differ from that of the balloon body 20. Such a balloon 2 can be produced, for example, by placing a parison obtained through extrusion molding in a mold with a groove and then blow-molding it. The preferred producing methods for the balloon will be described later.

The balloon 2 may have an inner protrusion part that projects inward from the inner surface of the balloon body 20 in the radial direction y 1. The protrusion part 28 and the inner protrusion part are preferably provided in the same position in the circumferential direction z1. The inner protrusion part is preferably integrally formed with the balloon body 20 and the protrusion part 28, and the inner protrusion part is preferably formed from the same material as the balloon body 20. Alternatively, when there is a certain degree of compatibility with the material used to form the balloon body 20, the material for forming the inner protrusion part may differ from that of the balloon body 20.

As shown in FIG. 3, the protrusion part 28 may be provided as a single protrusion part in the circumferential direction z1, or as shown in FIG. 4, the protrusion part 28 may be provided as a plurality of protrusions in the circumferential direction z1. When the balloon 2 has a plurality of protrusion parts 28 in the circumferential direction z1, each of the plurality of protrusion parts 28 is preferably spaced apart from each other in the circumferential direction z1, and more preferably arranged at equal intervals in the circumferential direction z1. The spacing distance is preferably longer than the maximum circumferential length of the protrusion part 28.

In the present invention, the protrusion part 28 is a portion that is formed to be thicker outward in the radial direction y1 than the film thickness at a predetermined position of the balloon body 20. The predetermined position refers to a position A, which is opposite in the circumferential direction z1 to an outer end 28T of the protrusion part 28 in the radial direction y1, when one protrusion part 28 is provided as shown in FIG. 3, and refers to a position B, which corresponds to the midpoint of the outer ends 28T in the circumferential direction z1 of the adjacent protrusion parts 28 in the circumferential direction z1, when a plurality of protrusion parts 28 are provided as shown in FIG. 4.

The maximum height of the protrusion part 28 in the radial direction y1 is preferably 1.2 times or more the film thickness at the predetermined position of the balloon body 20, more preferably 1.5 times or more, even more preferably 2 times or more, and may be 100 times or less, 50 times or less, 30 times or less, and 10 times or less is acceptable. The protrusion part 28 with a maximum height in the radial direction y1 in the above range allows for easier incision to an appropriate depth in the stenosis area, thereby facilitating crack formation.

The protrusion part 28 may have any cross-sectional shape in a cross-section perpendicular to the longitudinal axis direction x1, such as, for example, a triangle, a square, a polygon, a semi-circle, part of a circle, an approximate circle, a fan shape, a wedge shape, a convex shape, a spindle shape, and combinations thereof. Triangles, squares, and polygons shall include those with clear comer vertices and straight edges, as well as so-called rounded polygons with rounded corners and those with curved edges at least partly. Alternatively, the cross-sectional shape of the protrusion part 28 may be an irregular shape with concavity and convexity, chips, or the like.

When the protrusion part 28 is formed in a linear or dot shape, the protrusion part 28 is preferably arranged to extend along the longitudinal axis direction x1. Alternatively, the protrusion part 28 may be arranged to extend in a helical shape around the longitudinal axis.

A ratio Ra₁/Ra₂ of a surface roughness Ra₁ of the protrusion part 28, measured along a reference length in a direction parallel to the longitudinal axis direction x1, to a surface roughness Ra₂ of the protrusion part 28, measured along a reference length in a direction perpendicular to the longitudinal axis direction x1, is greater than 1.

The surface roughness is the arithmetic mean roughness Ra of the roughness curve over a reference length, and the reference length is 100 μm. The arithmetic mean roughness Ra corresponds to the arithmetic mean roughness Ra as defined in JIS B 0601 (2001). For measurement, for example, a laser microscope with white light interferometry VK-X3000 manufactured by Keyence Corporation can be used. For example, in the region R shown in FIG. 2, the direction parallel to the longitudinal axis direction x1 of the protrusion part 28 is the direction indicated by arrow a1, and the direction perpendicular to the longitudinal axis direction x1 of the protrusion part 28 is the direction indicated by arrow a2 in FIG. 2 to FIG. 4.

The surface roughness Ra₁ of the protrusion part 28 in the direction parallel to the longitudinal axis direction x1 can be obtained by measuring a roughness curve over a reference length of 100 μm in the direction of the arrow a1 and calculating the arithmetic average roughness of the roughness curve. At this time, the position in the circumferential direction z1 of the 100 μm reference length may be any position between a base end 28B and an outer end 28T of the protrusion part 28. The base end 28B of the protrusion part 28 is the part of the protrusion part 28 where the thickness of the radial direction y1 starts to exceed the film thickness at the above-describe predetermined position of the balloon body 20.

The surface roughness Ra₂ of the protrusion part 28 in the direction perpendicular to the longitudinal axis direction x1 can be obtained by measuring a roughness curve over a reference length of 100 μm in the direction of the arrow a2 and calculating the arithmetic average roughness of the roughness curve. At this time, the roughness curve for a reference length of 100 μm may be measured in the direction perpendicular to the longitudinal axis direction x1 from the base end 28B of the protrusion part 28, or the roughness curve for a reference length of 100 μm may be measured in the direction perpendicular to the longitudinal axis direction x1 from the outer end 28T, or the roughness curve for a reference length of 100 μm may be measured in the direction perpendicular to the longitudinal axis direction x1 at any position between the base end 28B and the outer end 28T. The surface length of the protrusion part 28 from the base end 28B to the outer end 28T in the direction perpendicular to the longitudinal axis direction x1 is preferably 100 μm or longer.

FIG. 5 shows an example of a roughness curve measured to determine Ra₁, and FIG. 6 shows an example of a roughness curve measured to determine Ra₂. From FIG. 5 and FIG. 6, it can also be understood that the surface roughness Ra₁ in the direction parallel to the longitudinal direction x1 of the protrusion part 28 is greater than the surface roughness Ra₂ measured for a refence length in the direction perpendicular to the longitudinal axis direction x1 of the protrusion part 28.

The protrusion part 28 can dilate the stenotic portion by embedding into it to form cracks when the balloon 2 delivered to the stenotic portion is inflated. At this time, the greater the surface roughness of the protrusion part 28 in the direction parallel to the longitudinal axis direction x1 of the balloon 2, i.e., the direction parallel to the balloon 2's travel direction in the blood vessel, the greater the resistance of the protrusion part 28 to the balloon 2's travel direction, and thus, the protrusion part 28 can improve the non-slip performance, which prevents it from deviating from the intended position. On the other hand, the smaller the surface roughness of the protrusion part 28 in the direction perpendicular to the longitudinal axis direction x1 of the balloon 2, i.e., the direction in which the protrusion part 28 enters the stenosis, the smaller the resistance of the protrusion part 28 against penetration into the stenosis, and the easier it is for the protrusion part 28 to bite into the stenosis, so the scoring performance can be improved. Accordingly, it is possible to make a balloon with non-slip and scoring performance by making the ratio Ra₁/Ra₂ of the surface roughness Ra₁ of the protrusion part 28, measured along a reference length in the direction parallel to the longitudinal axis direction x1, to the surface roughness Ra₂ of the protrusion part 28, measured along a reference length in the direction perpendicular to the longitudinal axis direction x1, greater than 1.

As an example of a configuration where the ratio Ra₁/Ra₂ is greater than 1, the surface of the protrusion part 28 may be designed such that fine ridges and grooves extending in the direction perpendicular to the longitudinal axis direction x1 are alternately arranged. In such a configuration, when the roughness curve is measured in the direction parallel to the longitudinal axis direction x1, the maximum height corresponds to the length from the top of the ridges to the bottom of the grooves, allowing Ra₁ to increase. Additionally, in this configuration, since the ridges and grooves extend perpendicular to the longitudinal axis x1, when the roughness curve is measured in the direction perpendicular to the longitudinal axis direction x1, the roughness curve passes through portions of nearly the same height of the ridges or the same depth of the grooves, enabling Ra₂ to be reduced. However, configurations where the ratio Ra₁/Ra₂ is greater than 1 are not limited to the example above. For instance, such configurations may include any arrangement such as the one in which fine irregularities are more densely distributed in the direction parallel to the longitudinal axis direction x1 and less densely distributed in the direction perpendicular to the longitudinal axis direction x1.

The surface roughness Ra₁ in the direction parallel to the longitudinal axis direction x1 is preferably determined as the average of the arithmetic mean roughness obtained from roughness curves measured over a specified number of reference lengths at predetermined intervals in the direction perpendicular to the longitudinal axis direction x1, i.e., in the circumferential direction z1. This allows for reducing the impact of surface roughness variations due to positional differences in the circumferential direction z1. The predetermined interval may be, for example, 2 μm, and the specific number of reference lengths may be, for example, 31. In this case, the surface roughness over an area of 60 μm×100 μm can be obtained.

The surface roughness Ra₂ in the direction perpendicular to the longitudinal axis direction x1 is preferably determined as the average of the arithmetic mean roughness obtained from roughness curves measured over a specified number of reference lengths at predetermined intervals in the longitudinal axis direction x1. This allows for reducing the impact of surface roughness variations due to positional differences in the longitudinal axis direction x1. The predetermined interval may be, for example, 2 μm, and the specific number of reference lengths may be, for example, 31. In this case, the surface roughness over an area of 60 μm×100 μm can be obtained.

The surface roughness Ra₁ in the direction parallel to the longitudinal axis direction x1 and the surface roughness Ra₂ in the direction perpendicular to the longitudinal axis direction x1 may be compared at the same position in the longitudinal axis direction x1, or at different positions in the longitudinal axis direction x1, but they are preferably compared at the same position in the longitudinal axis direction x1. Here, the same position means that the measurement areas measured for a predetermined number of reference lengths at a predetermined interval overlap. By comparing the surface roughness Ra₁ in the direction parallel to the longitudinal axis direction x1 and the surface roughness Ra₂ in the direction perpendicular to the longitudinal axis direction x1 at the same position in the longitudinal axis direction x1, the ratio Ra₁/Ra₂ being greater than 1 allows for the balloon 2 with improved non-slip performance and scoring performance of the protrusion part 28 at the position. For example, the respective surface roughness may be compared in the straight tubular part 23. When the Ra₁/Ra₂ in the straight tubular part 23 is greater than 1, the protrusion part 28 provided in the straight tubular part 23 can enhance the non-slip performance and scoring performance of the balloon 2, enabling more efficient dilation of the stenotic portion. When a plurality of protrusion parts 28 are provided, it suffices to measure the surface roughness of any one of the plurality of protrusion parts 28.

The ratio Ra₁/Ra₂ is preferably 1.2 or more, more preferably 1.5 or more, and may be 1.8 or more, 2 or more, 2.2 or more, 2.4 or more, and 2.5 or more; and preferably 5 or less, more preferably 4 or less, and even more preferably 3 or less. The ratio within the above range allows the balloon 2 to achieve improved non-slip performance and scoring performance due to the protrusion part 28.

As shown in FIG. 4, when multiple protrusion parts 28 are provided, the ratio Ra₁Ra₂ for all the protrusion parts 28 preferably falls within the above range. This ensures that the stenotic portion can be dilated more efficiently.

As shown in FIG. 2 to FIG. 4, in a cross-section perpendicular to the longitudinal axis direction x1, the protrusion part 28 preferably has a tip region 28t including an outer end 28T in the radial direction y1 and base region 28b located inward from the tip region 28t in the radial direction y1, a surface roughness of the base region 28b is preferably smaller than a surface roughness of the tip region 28t where a surface roughness of the protrusion part 28 is measured along a reference length in a direction parallel to the longitudinal axis direction x1, and a surface roughness of the base region 28b is preferably smaller than a surface roughness of the tip region 28t where a surface roughness of the protrusion part 28 is measured along a reference length in a direction perpendicular to the longitudinal axis direction x1.

The rougher surface roughness in the direction parallel to the longitudinal axis direction x1 increases the resistance of the protrusion part 28 to the balloon 2's travel direction. The rougher surface roughness of the tip region 28t, which first comes into contact with the stenotic portion, makes it difficult for the protrusion part 28 to deviate from its intended position at the start of penetration, so the non-slip performance can be further improved. Once positioning has been achieved by the tip region 28t, the smaller the surface roughness of the base region 28b, which subsequently penetrates the stenotic portion following the tip region 28t, the easier it is for the entire protrusion part to penetrate the stenotic portion.

The surface roughness in the direction perpendicular to the longitudinal axis direction x1 affects the resistance of the protrusion part 28 as it penetrates the stenotic portion. Specifically, while the tip region 28t, which firsts penetrates the stenotic portion, can penetrate with relatively low resistance to pushing force, the base region 28b, which penetrates at the later stage, encounters greater resistance to pushing force, making it difficult for the entire protrusion part 28 to penetrate the stenosis. However, the surface roughness of the base region 28b smaller than the surface roughness of the tip region 28t when the surface roughness is measured in the direction perpendicular to the longitudinal axis direction x1 can reduce the frictional resistance of the base region 28b. This enables the entire protrusion part 28 to penetrate the stenosis. As a result, the scoring performance can be further improved.

The surface roughness of the tip region 28t and the base region 28b can also be measured using the same method as that for measuring the surface roughness of the protrusion part 28 described above. For defining the ranges of the tip region 28t and the base region 28b, for example, a straight line parallel to the longitudinal axis direction x1 can be drawn on the surface of the protrusion part 28 through the midpoint of a segment perpendicular to the longitudinal axis direction x1, extending from the base end 28B to the outer end 28T, and the outer side of the straight line in the radial direction y1 can be the tip region 28t and the base side of the straight line in the radial direction y1 can be the base region 28b. Alternatively, the straight line may pass through a point closer to the tip side than the midpoint of the segment perpendicular to the longitudinal axis direction x1, extending from the base end 28B to the outer end 28T, or it may pass through a point closer to the base side than the midpoint.

The surface roughness of the tip region 28t in the direction parallel to the longitudinal axis direction x1 can be determined by measuring the surface roughness near the outer end 28T of the protrusion part 28 in the radial direction y1, and preferably determined as the average of arithmetic mean roughness values obtained from roughness curves measured along a specified number of reference lengths at predetermined intervals in the circumferential direction z1. The predetermined interval can be, for example, 2 μm, and the specified number of reference lengths can be 31. In this case, the length of the surface of the tip region 28t in the direction perpendicular to the longitudinal axis direction x1, i.e., in the circumferential direction z1, is preferably 60 μm or longer. Alternatively, when the length of the surface of the tip region 28t in the direction perpendicular to the longitudinal axis direction x1, i.e., in the circumferential direction z1, is less than 60 μm, it is also possible, for example to use narrower predetermined intervals or reduced specified number of reference lengths.

The surface roughness of the base region 28b in the direction parallel to the longitudinal axis direction x1 can be determined by measuring the surface roughness near the base end 28B of the protrusion part 28 in the radial direction y1, and preferably determined as the average of arithmetic mean roughness values obtained from roughness curves measured along a specified number of reference lengths at predetermined intervals in the circumferential direction z1. The predetermined interval can be, for example, 2 μm, and the specified number of reference lengths can be 31. In this case, the length of the surface of the base region 28b in the direction perpendicular to the longitudinal axis direction x1, i.e., in the circumferential direction z1, is preferably 60 μm or longer. Alternatively, when the length of the surface of the base region 28b in the direction perpendicular to the longitudinal axis direction x1, i.e., in the circumferential direction z1, is less than 60 μm, it is also possible, for example to use narrower predetermined intervals or reduced specified number of reference lengths.

The surface roughness of the tip region 28t in the direction perpendicular to the longitudinal axis direction x1 can be determined by measuring the roughness curve over a reference length of 100 μm in the direction perpendicular to the longitudinal axis direction x1, starting from the vicinity of the outer end 28T of the protrusion part 28 in the radial direction y1. In this case, the surface roughness is preferably determined as the average of the arithmetic mean roughness values obtained from roughness curves measured along a specified number of reference lengths at predetermined intervals in the longitudinal axis direction x1.

The surface roughness of the base region 28b in the direction perpendicular to the longitudinal axis direction x1 can be determined by measuring the roughness curve over a reference length of 100 μm in the direction perpendicular to the longitudinal axis direction x1, starting from the vicinity of the base end 28B of the protrusion part 28 in the radial direction y1. In this case, the surface roughness is preferably determined as the average of the arithmetic mean roughness values obtained from roughness curves measured along a specified number of reference lengths at predetermined intervals in the longitudinal axis direction x1.

When measuring the surface roughness in the direction perpendicular to the longitudinal axis direction x1 as described above, if the length from the base end 28B to the outer end 28T of the protrusion part 28 in the direction perpendicular to the longitudinal axis direction x1, i.e., in the circumferential direction z1, is shorter than twice the reference length of 100 μm, i.e., shorter than 200 μm, the measurement areas of the base region 28b and the tip region 28t will partially overlap in the direction perpendicular to the longitudinal axis direction x1, but the roughness of the tip region 28t and the base region 28b can be obtained by measuring the surface roughness in the above manner.

Alternatively, if he length from the base end 28B to the outer end 28T of the protrusion part 28 in the direction perpendicular to the longitudinal axis direction x1, i.e., in the circumferential direction z1, is longer than twice the reference length of 100 μm, i.e., longer than 200 μm, the surface roughness of the tip region 28t in the direction perpendicular to the longitudinal axis direction x1 can be obtained by measuring a roughness curve over a reference length of 100 μm in the direction perpendicular to the longitudinal axis direction x1 at any position on the outer end 28T side than the point 100 μm away from the base end 28B to the outer end 28T side in the direction perpendicular to the longitudinal axis direction x1 The starting and ending points of the reference length may be located at any point between the point 100 μm away from the base end 28B and the outer end 28T in the direction perpendicular to the longitudinal axis direction x1, i.e., in the circumferential direction z1.

The surface roughness of the tip region 28t and the base region 28b may be compared at the same position in the longitudinal axis direction x1, or at different positions in the longitudinal axis direction x1, but they are preferably compared at the same position in the longitudinal axis direction x1. Here, the same position can refer to exactly the same position in the longitudinal axis direction x1, or it may mean that the measurement areas in the tip region 28t and the base region 28b overlap at least partially in the longitudinal axis direction x1. The surface roughness of the base region 28b smaller than the surface roughness of the tip region 28t, both of which are located at the same position in the longitudinal axis direction x1, allows the protrusion part 28 at the position to achieve the above-described effect. For example, the surface roughness of each region may be compared in the straight tubular part 23.

The surface roughness of the base region 28b in the direction parallel to the longitudinal axis direction x1 is preferably 0.9 times or less the surface roughness of the tip region 28t in the direction parallel to the longitudinal axis direction x1, more preferably 0.8 times or less, even more preferably 0.6 times or less, and may be 0.5 times or less, and 0.4 times or less; and preferably 0.01 times or more, more preferably 0.05 times or more, and even more preferably 0.1 times or more. The surface roughness in the above range makes it easier to obtain the balloon 2 with improved non-slip performance due to the tip region 28t.

The surface roughness of the base region 28b in the direction perpendicular to the longitudinal axis direction x1 is preferably 0.9 times or less the surface roughness of the tip region 28t in the direction perpendicular to the longitudinal axis direction x1, more preferably 0.8 times or less, even more preferably 0.6 times or less, and may be 0.5 times or less, and 0.4 times or less; and preferably 0.01 times or more, more preferably 0.05 times or more, and even more preferably 0.1 times or more. The surface roughness in the above range enables the balloon 2 in which the entire protrusion part 28 can easily penetrate the stenotic portion.

In the case where multiple protrusion parts 28 are provided as shown in FIG. 4, the tip regions 28t and the base regions 28b of all the protrusion parts 28 preferably satisfy the above relationship. This makes the effective dilation of the stenotic portion easier.

In a cross-section perpendicular to the longitudinal axis direction x1, the protrusion part 28 has the tip region 28t including the outer end 28T in the radial direction y1 and the base region 28b located inward from the tip region 28t in the radial direction y1; a ratio Ra₁₍ₜᵢₚ₎/Ra₂₍ₜᵢₚ₎ of a surface roughness Ra₁₍ₜᵢₚ₎, where the surface roughness of the tip region 28t is measured along a reference length in the direction parallel to the longitudinal axis direction x1, to a surface roughness Ra₂₍ₜᵢₚ₎, where the surface roughness of the tip region 28t is measured along a reference length in the direction perpendicular to the longitudinal axis direction x1, is preferably greater than 1; and a ratio Ra_{1(base)}/Ra_{2(base)} of a surface roughness Ra_{1(base)}, where the surface roughness of the base region 28b is measured along a reference length in the direction parallel to the longitudinal axis direction x1, to a surface roughness Ra_{2(base)}, where the surface roughness of the base region 28b is measured along a reference length in the direction perpendicular to the longitudinal axis direction x1, is preferably greater than 1.

By having the surface roughness in the direction parallel to the longitudinal axis direction x1 greater than the surface roughness in the direction perpendicular to the longitudinal axis direction x1 in both the tip region 28t and the base region 28b, it becomes easier to obtain the balloon 2 that improves both non-slip performance and scoring performance.

When the surface roughness of the base region 28b in the direction perpendicular to the longitudinal axis direction xl is smaller than the surface roughness of the tip region 28t in the direction perpendicular to the longitudinal axis direction x1, a surface roughness of the balloon body 20 is preferably greater than the surface roughness of the base region 28b where the surface roughness of the base region 28b and the surface roughness of the balloon body 20 are measured along a reference length in the direction perpendicular to the longitudinal axis direction x1. While the balloon 2 in accordance with embodiments of the present invention allows the protrusion part 28 to easily penetrate the stenotic portion, the greater surface roughness of the balloon body 20 in the direction perpendicular to the longitudinal axis direction x1 than in the base region 28b can prevent the balloon body 20 itself from penetrating the stenotic portion, thereby preventing the cracks from becoming unnecessarily wide.

The surface roughness of the balloon body 20 in the direction perpendicular to the longitudinal axis direction x1 is preferably 1.2 times or more the surface roughness of the base region 28b in the direction perpendicular to the longitudinal axis direction x1, more preferably 1.5 times or more, even more preferably 2 times or more, particularly preferably 3 times or more, and preferably 10 times or less, more preferably 9 times or less, and even more preferably 8 times or less.

When the surface roughness of the base region 28b in the direction perpendicular to the longitudinal axis direction x1 is smaller than the surface roughness of the tip region 28t in the direction perpendicular to the longitudinal axis direction x1, the surface roughness of the balloon body 20 is preferably greater than the surface roughness of the tip region 28t where the surface roughness of the tip region 28t and the surface roughness of the balloon body 20 are measured along a reference length in the direction perpendicular to the longitudinal axis direction x1. The surface roughness of the balloon body 20 being even greater than the surface roughness of the tip region 28t, which is greater than that of the base region 28b, makes it easier to prevent the balloon body 20 itself from penetrating the stenotic portion.

This facilitates allowing only the protrusion part 28 to penetrate the stenotic portion and create the desired incision.

The surface roughness of the balloon body 20 in the direction perpendicular to the longitudinal axis direction x1 is preferably 1.05 times or more the surface roughness of the tip region 28t in the direction perpendicular to the longitudinal axis direction x1, more preferably 1.1 times or more, even more preferably 1.2 times or more, particularly preferably 1.5 times or more, and preferably 8 times or less, more preferably 7 times or less, and even more preferably 6 times or less.

When the surface roughness of the base region 28b in the direction parallel to the longitudinal axis direction x1 is smaller than the surface roughness of the tip region 28t in the direction parallel to the longitudinal axis direction x1, the surface roughness of the balloon body 20 is preferably smaller than the surface roughness of the tip region 28t where the surface roughness of the tip region 28t and the surface roughness of the balloon body 20 are measured along a reference length in the direction parallel to the longitudinal axis direction x1. While since greater surface roughness in the direction parallel to the longitudinal axis direction x1 can improve the non-slip performance, the large surface roughness of the tip region 28t can ensure the non-slip performance of the protrusion part 28, the small surface roughness of the balloon body 20, which has a large contact area with the vessel wall during delivery, in the direction parallel to the longitudinal axis direction x1 enhances the insertion performance of the balloon 2 within the vascular lumen during delivery.

The surface roughness of the balloon body 20 in the direction parallel to the longitudinal axis direction x1 is preferably 0.6 times or less the surface roughness of the tip region 28t in the direction parallel to the longitudinal axis direction x1, more preferably 0.5 times or less, even more preferably 0.4 times or less, and may be 0.3 times or less, 0.2 times or less; and preferably 0.03 times or more, more preferably 0.05 times or more, even more preferably 0.08 times or more, and may be 0.1 times or more.

When the surface roughness of the base region 28b in the direction parallel to the longitudinal axis direction x1 is smaller than the surface roughness of the tip region 28t in the direction parallel to the longitudinal axis direction x1, the surface roughness of the balloon body 20 is preferably smaller than the surface roughness of the base region 28b where the surface roughness of the base region 28b and the surface roughness of the balloon body 20 are measured along a reference length in the direction parallel to the longitudinal axis direction x1. The surface roughness of the balloon body 20 being even smaller than the surface roughness of the base region 28b, which is smaller than that of the tip region 28t, can further improve the insertion performance of the balloon 2 within the vascular lumen during delivery.

The surface roughness of the balloon body 20 in the direction parallel to the longitudinal axis direction x1 is preferably 0.99 times or less the surface roughness of the base region 28b in the direction parallel to the longitudinal axis direction x1, more preferably 0.8 times or less, even more preferably 0.7 times or less, and preferably 0.3 times or more, more preferably 0.4 times or more, even more preferably 0.5 times or more.

Materials forming the balloon body 20 and the protrusion part 28 include, for example, polyolefin-based resin such as polyethylene, polypropylene, ethylene-propylene copolymer; polyester-based resin such as polyethylene terephthalate and polyester elastomer; polyurethane-based resin such as polyurethane and polyurethane elastomer; polyphenylene sulfide-based resin; polyamide-based resin such as polyamide and polyamide elastomer; fluorine-based resin; silicone-based resin; and natural rubber such as latex rubber. Only one of these may be used, or two or more may be used in combination. Of these, polyamide-based resin, polyester-based resin, and polyurethane-based resin are preferably used, more preferably polyamide-based resin such as nylon 12 and nylon 11, and particularly preferably nylon 12. From the viewpoint of thinning and flexibility of the balloon body 20, elastomer resin is preferably used, and polyamide elastomer such as polyamide ether elastomer is preferably used.

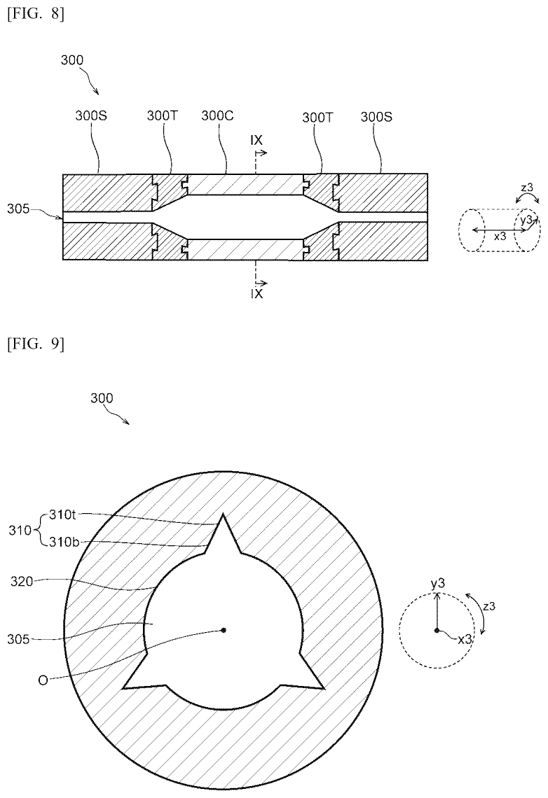

Next, a method for producing the balloon 2 will be explained with reference to FIG. 7 to FIG. 9. FIG. 7 is a perspective view of a parison in accordance with one embodiment of the present invention before inflation. FIG. 8 is a cross-sectional view in the longitudinal axis direction of a mold in accordance with one embodiment of the present invention, and FIG. 9 is a IX-IX cross-sectional view of FIG. 8.

The balloon 2 can be produced by arranging a parison 200 in a mold 300 and blow-molding the parison 200.

As shown in FIG. 7, the parison 200 is a tubular member made of resin and having a lumen 205. The parison 200 is made, for example, by extrusion molding. The parison 200 has a first end 201 and a second end 202, and extends in a longitudinal axis direction x2 from the first end 201 to second end 202. The parison 200 has a radial direction y2 and a circumferential direction z2, similar to the balloon 2.

The cross-sectional shape perpendicular to the longitudinal axis direction x2 of the parison 200 may be uniform in the longitudinal axis direction x2. This improves the productivity of the parison 200. Alternatively, although not shown in the figures, the cross-sectional shape perpendicular to the longitudinal axis direction x2 of the parison 200 may differ depending on the position in the longitudinal axis direction x2. For example, the outer diameter of a portion of the parison 200 in the longitudinal axis direction x2 may be larger than that of other portions, and the portion with the larger outer diameter may be molded to become the straight tubular part 23 of the balloon 2. In order to manufacture a parison 200 with an outer diameter that differs depending on the position in the longitudinal axis direction x2, blow molding may be performed using a mold in advance.

As shown in FIG. 7, the parison 200 before inflation may have a protrusion part 208 that is thicker outwardly in the radial direction y2. By making the protrusion part 208 contact a groove part 310 of the mold 300, which are described below, it becomes easier to form the protrusion part 28 of the balloon 2.

As shown in FIG. 7, the protrusion parts 208 may be provided in multiple locations in the circumferential direction z2, or, although not shown in the figures, the protrusion part 208 may be provided in a single location in the circumferential direction z2. In the case where the protrusion parts 208 are provided in multiple locations in the circumferential direction z2, the multiple protrusion parts 208 are preferably space apart from each other in the circumferential direction z2, and more preferably arranged at equal intervals in the circumferential direction z2.

As for the materials constituting the parison 200, reference can be made to the above description of the resin used for forming the balloon body 20 and the protrusion part 28.

As shown in FIG. 8, the mold 300 has a longitudinal axis direction x3, a radial direction y3, and a circumferential direction z3, and has an inner cavity 305 extending in the longitudinal axis direction x3 through which the parison 200 is to be inserted. Preferably, a part of the parison 200 in the longitudinal axis direction x2 is positioned within the inner cavity 305 of the mold 300. The longitudinal axis direction x2 of the parison 200 preferably aligns with the longitudinal axis direction x3 of the mold 300. This facilitates the placement of the parison 200 within the inner cavity 305 of the mold 300.

The mold 300 preferably has, in the longitudinal axis direction x3, a mold straight tubular part 300C, which forms the straight tubular part 23 of the balloon 2, two mold tapered parts 300T, positioned on both sides of the mold straight tubular part 300C to form the tapered parts of the balloon 2, and two mold sleeve parts 300S, positioned further side of the mold straight tubular part 300C than the mold tapered parts 300T to form the sleeve parts of the balloon 2. This allows the mold straight tubular part 300C to form the straight tubular part 23 of the balloon 2, the mold tapered parts 300T to form the proximal tapered part 22 and distal tapered part 24 of the balloon 2, and the mold sleeve parts 300S to form the proximal sleeve part 21 and distal sleeve part 25.

The mold 300 may be composed of a single component or multiple components. As shown in FIG. 8, it may be configured by connecting multiple mold components along the longitudinal axis direction x3. For example, the mold straight tubular part 300C, the mold tapered parts 300T, and the mold sleeve parts 300S may each be separate mold components, and they may be connected to each other along the longitudinal axis direction x3. In addition, the mold 300 may be divisible in the radial direction y3.

As shown in FIG. 9, the inner cavity 305 of the mold 300 preferably consists of a groove part 310 recessed outwardly in the radial direction y3 and extending in the longitudinal axis direction x3 and a cylindrical wall part 320 other than the groove part 310. The balloon 2 with the protrusion part 28 can be produced by allowing the parison 200 to enter the groove part 310 of the mold 300 and introducing fluid into the lumen 205 of the parison 200 to blow-mold the parison 200.

The groove part 310 is provided in the mold straight tubular part 300C of the mold 300. This allows the protrusion part 28 to be formed in the straight tubular part 23 of the balloon 2, thereby enhancing the efficiency of stenosis incision by the balloon 2.

The groove part 310 may be provided in at least one of the two mold tapered parts 300T of the mold 300. This allows the protrusion part 28 to be formed on the proximal tapered part 22 and/or distal tapered part 24 of the balloon 2, improving non-slip performance of the balloon 2 against the stenosis. When the groove part 310 is provided in the mold tapered parts 300T, the depth of the groove part 310 provided in the mold tapered parts 300T is preferably no greater than the depth of the groove part 310 provided in the mold straight tubular part 300C. This allows the height of the protrusion part 28 formed on the proximal tapered part 22 and/or distal tapered part 24 of the balloon 2 to be no greater than the height of the protrusion part 28 formed on the straight tubular part 23, improving the insertion performance of the balloon 2. If the groove part 310 is not provided in the mold tapered parts 300T, the protrusion part 28 will not be formed on the proximal tapered part 22 and/or distal tapered part 24 of the balloon 2, or the height of the protrusion part 28 will be reduced. further improving the insertion performance of the balloon 2. In this case, an inner protrusion part may be formed in the areas where the protrusion part 28 is either not formed or is formed with a reduced height.

The groove part 310 may or may not be provided in the mold sleeve parts 300S of the mold 300. When the groove part 310 is provided in the mold sleeve parts 300S, the depth of the groove part 310 provided in the mold sleeve parts 300S is preferably smaller than the depth of the groove part 310 provided in the mold straight tubular part 300C. This allows the height of the protrusion part 28 formed on the proximal sleeve part 21 and/or distal sleeve part 25 to be lower than the protrusion part formed on the straight tubular part 23, improving the insertion performance of the balloon 2. If the groove part 310 is not provided in the mold sleeve parts 300S, the protrusion part 28 will not be formed on the proximal sleeve part 21 and/or distal sleeve part 25 of the balloon 2, further improving the insertion performance of the balloon 2. In this case, an inner protrusion part may be formed in the areas where the protrusion part 28 is either not formed or is formed with a reduced height.

One configuration for increasing the ratio Ra₁/Ra₂ of the surface roughness of the protrusion part 28 of the balloon 2 to greater than 1 involves alternately forming fine ridges and grooves extending in the direction perpendicular to the longitudinal axis direction x1 on the surface of the protrusion part 28. As a method for producing this configuration, for example, there is a method of polishing the inner cavity 305 of the groove part 310 or the mold 300 in a direction perpendicular to the longitudinal axis direction x3, i.e., in the circumferential direction z3, thereby forming fine polishing marks in the direction perpendicular to the longitudinal axis direction x3, i.e., in the circumferential direction z3.

A method for producing the balloon 2 in which the surface roughness differs between the base region 28b and the tip region 28t of the protrusion part 28 includes, for example, forming polishing marks on the mold 300 such that the surface roughness of the groove base region 310b and the groove tip region 310t of the groove part 310 differs.

A method for producing the balloon 2 such that the surface roughness of the balloon body 20 is greater than that of the protrusion part 28 when measured over a reference length in the direction perpendicular to the longitudinal axis direction x1, and the surface roughness of the balloon body 20 is smaller than that of the protrusion part 28 when measured over a reference length in the direction parallel to the longitudinal axis direction x1, includes, for example, polishing a cylindrical wall part 320 that forms the balloon body 20 in the longitudinal axis direction x3 to form fine polishing marks in the longitudinal axis direction x3 Materials constituting the mold 300 is preferably metal, and more preferably, iron, copper, aluminum, or an alloy of these. Stainless steel is an example of an iron alloy, brass is an example of a copper alloy, and duralumin is an example of an aluminum alloy.

### 2. Balloon Catheter

A balloon catheter 1 in accordance with embodiments of the present invention is provided with the above-described balloon 2 for a balloon catheter. As described in the section “1. Balloon for balloon catheter,” the balloon 2 is connected to the distal end part of the distal shaft 31, as shown in FIG. 1.

FIG. 1 illustrates a so-called rapid-exchange type balloon catheter 1, which has a guidewire port 50 located midway between the distal and proximal sides of the shaft 30 and has an inner shaft 60 that functions as a guidewire lumen from the guidewire port 50 to the distal side of the shaft 30. The balloon catheter 1 preferably has a distal shaft 31 and a proximal shaft 32, with the distal shaft 31 and the proximal shaft 32 being separate members. The proximal end part of the distal shaft 31 may be connected to the distal end part of the proximal shaft 32, thereby forming the shaft 30 that extends from the balloon 2 to the proximal end part of the balloon catheter 1. Alternatively, a single shaft 30 may extend from the balloon 2 to the proximal end part of the balloon catheter 1, or the distal shaft 31 and the proximal shaft 32 may each consist of multiple tube members.

The shaft 30 preferably has a fluid flow path and a guidewire lumen inside. To configure the shaft 30 to have the internal fluid flow path and the guidewire lumen, for example, the inner shaft 60 located inside the shaft 30 may function as the guidewire lumen, and the space between the shaft 30 and the inner shaft 60 may function as the fluid flow path. In such a configuration, preferably, the inner shaft 60 extends from the distal end of the shaft 30 and passes through the balloon 2, the distal side of the balloon 2 is connected to the inner shaft 60, and the proximal side of the balloon 2 is connected to the shaft 30.

The shaft 30 is preferably composed of resin, metal, or a combination of resin and metal. By using resin as the material for the shaft, flexibility and elasticity can be more easily imparted to the shaft 30. By using metal as the material for the shaft 30, the delivering performance of the balloon catheter 1 can be improved. Examples of resin used for the shaft 30 include polyamide-based resin, polyester-based resin, polyurethane-based resin, polyolefin-based resin, fluorine-based resin, polyvinyl chloride-based resin, silicone-based resin, natural rubber, and synthetic rubber. Any one of these may be used alone, or two or more may be used in combination. Examples of metal used for the shaft 30, include stainless steel such as SUS 304 and SUS 316, platinum, nickel, cobalt, chromium, titanium, tungsten, gold, Ni—Ti alloys, Co—Cr alloys, or combinations thereof. When the shaft 30 is composed of the distal shaft 31 and proximal shaft 32 as separate members, the distal shaft 31 may be, for example, made of resin, and the proximal shaft 32 may be made of metal. The shaft 30 may also have a layered structure using different materials or the same material.

The balloon 2 and the shaft 30 may be joined by adhesive bonding, welding, or by attaching a ring-shaped member at the point where the end of the balloon 2 and the shaft 30 overlap to swage them. Of these, the balloon 2 and the shaft 30 are preferably joined by welding. By welding the balloon 2 and the shaft 30, the bond between the balloon 2 and the shaft 30 is difficult to be released even when the balloon 2 is repeatedly inflated and deflated, easily increasing the strength of the bond between them.

The balloon catheter 1 is preferably provided with a tip member 70 at its distal end part. The tip member 70 may be provided at the distal end part of the balloon catheter 1 by being connected to the distal end part of the balloon 2 as a separate component from the inner shaft 60, or the inner shaft 60 extending distally beyond the distal end of the balloon 2 may function as the tip member 70.

A radiopaque marker 80 may be placed on the inner shaft 60 inside the balloon 2 at the location of the balloon 2 in the longitudinal axis direction x1, so that the position of the balloon 2 can be confirmed radiographically. The radiopaque marker 80 is preferably placed at a position corresponding to both ends of the straight tubular part 23 of the balloon 2, or may be placed at a position corresponding to the center of the straight tubular part 23 in the longitudinal axis direction x1.

A hub 5 may be provided at a proximal side of the shaft 30, and the hub 5 may be provided with a fluid inlet 6 that is connected to the flow channel of the fluid supplied to the interior of the balloon 2.

The shaft 30 and the hub 5 may be joined by, for example, adhesive bonding or welding. Of these, the shaft 30 and the hub 5 are preferably joined by adhesive bonding.

The adhesive bonding of the shaft 30 and hub 5 can increase the bonding strength of the shaft 30 and hub 5 to increase durability of the balloon catheter 1 when the materials forming the shaft 30 and hub 5 are different, for example, in a case where the shaft 30 is made of material having high flexibility and the hub 5 is made of material having high stiffness.

Although not shown in the figures, the present invention is also applicable to a so-called over-the-wire type balloon catheter that has a guidewire lumen extending from the distal end to the proximal end of the shaft. In the case of the over-the-wire type, the inflation lumen and the guidewire lumen preferably extend to a hub positioned at the proximal side, and the proximal openings of each lumen are preferably provided in the hub having a bifurcated structure.

In the case of the rapid-exchange type catheter, the outer wall of the distal shaft 31 and/or the proximal shaft 32 is preferably coated as appropriate, and more preferably, both the distal shaft 31 and the proximal shaft 32 are coated. In the case of the over-the-wire type catheter, the outer wall of the outer shaft is preferably coated as appropriate.

The coating can be a hydrophilic or hydrophobic coating, depending on the purpose, and can be applied by dipping the shaft 30 into a hydrophilic or hydrophobic coating agent, applying a hydrophilic or hydrophobic coating agent to the outer wall of the shaft 30, or coating the outer wall of the shaft 30 with a hydrophilic or hydrophobic coating agent. The coating agent may contain medical agents and additives.

Hydrophilic coating agents include hydrophilic polymers such as polyvinyl alcohol, polyethylene glycol, polyacrylamide, polyvinyl pyrrolidone, methyl vinyl ether maleic anhydride copolymer, and hydrophilic coating agents made of any combination thereof.

Hydrophobic coating agents include polytetrafluoroethylene (PTFE), ethylene-propylene fluoride (FEP), perfluoroalkoxy alkane (PFA), silicone oil, hydrophobic urethane resin, carbon coat, diamond coat, diamond-like carbon (DLC) coating, ceramic coating, and substances with low surface free energy terminated with an alkyl group or a perfluoroalkyl group.

The present application claims priority based on Japanese Patent Application No. 2022-133113 filed on Aug. 24, 2022. All the contents described in Japanese Patent Application No. 2022-133113 filed on Aug. 24, 2022 are incorporated herein by reference.

### DESCRIPTION OF REFERENCE SIGNS

1: balloon catheter
2: balloon for balloon catheter
5: hub
6: fluid inlet
20: balloon body
21: proximal sleeve part
22: proximal tapered part
23: straight tubular part
24: distal tapered part
25: distal sleeve part
28: protrusion part
28b: base region
28B: base end
28t: tip region
28T: outer end
30: shaft
31: distal shaft
32: proximal shaft
50: guidewire port
60: inner shaft
70: tip member
80: radiopaque marker
200: parison
201: first end of the parison
202: second end of the parison
205: lumen of the parison
208: protrusion part of the parison
300: mold
300C: mold straight tubular part
300S: mold sleeve parts
300T: mold tapered parts
305: inner cavity of the mold
310: groove part
310b: groove base region
300t: groove tip region
320: cylindrical wall part
a1: direction parallel to the longitudinal axis direction of the balloon
a2: direction perpendicular to the longitudinal axis direction of the balloon
x1: longitudinal axis direction of the balloon
y1: radial direction of the balloon
z1: circumferential direction of the balloon
x2: longitudinal axis direction of the parison
y2: radial direction of the parison
z2: circumferential direction of the parison
x3: longitudinal axis direction of the mold
y3: radial direction of the mold
z3: circumferential direction of the mold

1. A balloon for a balloon catheter having a longitudinal axis direction and a radial direction, comprising:
a balloon body having an outer surface and inner surface; and
a protrusion part that projects outward from the outer surface of the balloon body in the radial direction and extends in the longitudinal axis direction, wherein
the balloon body and the protrusion part are composed of the same material; and
a ratio Ra₁/Ra₂ of a surface roughness Ra₁ of the protrusion part, measured along a reference length in a direction parallel to the longitudinal axis direction, to a surface roughness Ra₂ of the protrusion part, measured along a reference length in a direction perpendicular to the longitudinal axis direction, is greater than 1.

2. The balloon for a balloon catheter according to claim 1, wherein
in a cross-section perpendicular to the longitudinal axis direction, the protrusion part has a tip region including an outer end in the radial direction and a base region located inward from the tip region in the radial direction;
a surface roughness of the base region is smaller than a surface roughness of the tip region where a surface roughness of the protrusion part is measured along a reference length in a direction parallel to the longitudinal axis direction; and
a surface roughness of the base region is smaller than a surface roughness of the tip region where a surface roughness of the protrusion part is measured along a reference length in a direction perpendicular to the longitudinal axis direction.

3. The balloon for a balloon catheter according to claim 1, wherein
in a cross-section perpendicular to the longitudinal axis direction, the protrusion part has a tip region including an outer end in the radial direction and a base region located inward from the tip region in the radial direction,
a ratio Ra₁₍ₜᵢₚ₎/Ra₂₍ₜᵢₚ₎ of a surface roughness Ra₁₍ₜᵢₚ₎, where a surface roughness of the tip region is measured along a reference length in a direction parallel to the longitudinal axis direction, to a surface roughness Ra₂₍ₜᵢₚ₎, where a surface roughness of the tip region is measured along a reference length in a direction perpendicular to the longitudinal axis direction, is greater than 1; and
a ratio Ra_{1(base)}/Ra_{2(base)} of a surface roughness Ra_{1(base)}, where a surface roughness of the base region is measured along a reference length in a direction parallel to the longitudinal axis direction, to a surface roughness Ra_{2(base)}, where a surface roughness of the base region is measured along a reference length in a direction perpendicular to the longitudinal axis direction, is greater than 1.

4. The balloon for a balloon catheter according to claim 2, wherein a surface roughness of the balloon body is greater than a surface roughness of the base region where the surface roughness of the base region and the surface roughness of the balloon body are measured along a reference length in a direction perpendicular to the longitudinal axis direction.

5. The balloon for a balloon catheter according to claim 2, wherein a surface roughness of the balloon body is greater than a surface roughness of the tip region where the surface roughness of the tip region and the surface roughness of the balloon body are measured along a reference length in a direction perpendicular to the longitudinal axis direction.

6. The balloon for a balloon catheter according to claim 2, wherein a surface roughness of the balloon body is smaller than a surface roughness of the tip region where the surface roughness of the tip region and the surface roughness of the balloon body are measured along a reference length in a direction parallel to the longitudinal axis direction.

7. The balloon for a balloon catheter according to claim 2, wherein a surface roughness of the balloon body is smaller than a surface roughness of the base region where the surface roughness of the base region and the surface roughness of the balloon body are measured along a reference length in a direction parallel to the longitudinal axis direction.

8. A balloon catheter comprising the balloon for a balloon catheter according to claim 1.

9. The balloon for a balloon catheter according to claim 1, wherein the ratio Ra₁/Ra₂ is 1.2 or greater.

10. The balloon for a balloon catheter according to claim 2, wherein the reference length of the base region is 100 μm from the lower end of the base region toward the outer end of the tip region, and
the reference length of the tip region is 100 μm from the outer end of the tip region toward the lower end of the base region.

11. The balloon for a balloon catheter according to claim 1, wherein the surface of the protrusion part comprises a plurality of ridges and grooves, each extending in the direction perpendicular to the longitudinal axis direction and alternately arranged, so that the ratio Ra₁/Ra₂ is greater than 1.
